SEQUENCE LISTING (16 sequences)

SEQ ID NO: 1 - DNA, 673 bp, Artificial Sequence
  synthetic nucleotide sequence - Guayule Contig 19031
  misc_feature (201)..(209): n is A, C, G or T
gagttaacaaaatggaaactattgaatacgatggttcttcttcgatctcttcatcatcatcgtctcaaaccgagactactgaacgactacccacaccatcgtcaacaaaccactcgaaaagaaaatccggaaggaagaagtttaaagaaacgagacacccggtctatagaggggtccggttgagaaacgggtctaaatggnnntgcgnngttcgtgagcctagtaagaaatcaagaatttggttaggaacatttccaactccggaaatggcagctagggcttatgatgcagcaaccctagccttgcatggtgatgactcaccacttaactttgctgactcggcccgtctaattagacgggccaagtcaacttctgcttctgatgttcaggaggcagcccttgaggcggctcttgcatttgggtcaaatcatcatgttgaccctaagccgtcttcttcatcttcagcgtgttttcagatggagaaaggtgaggaaaacttgacagttgatgtggcgcatacatcatttgtggacgaagagttgttgtttaatacgccgagtttttataacagtatggcggagggattggtgattacaccaccgggtatgaaaaaagggtttgattggagtgatgattgtgcctccaatatggatttgactctatacctgcgcgg

SEQ ID NO: 2 - protein, 220 aa, Artificial Sequence
  synthetic peptide sequence - Guayule Contig 19031
  MISC_FEATURE (64)..(64): Xaa is any amino acid
  MISC_FEATURE (66)..(66): Xaa is any amino acid
METIEYDGSSSISSSSSSQTETTERLPTPSSTNHSKRKSGRKKFKETRHPVYRGVRLRNGSKWXCXVREPSKKSRIWLGTFPTPEMAARAYDAATLALHGDDSPLNFADSARLIRRAKSTSASDVQEAALEAALAFGSNHHVDPKPSSSSSACFQMEKGEENLTVDVAHTSFVDEELLFNTPSFYNSMAEGLVITPPGMKKGFDWSDDCASNMDLTLYLR

SEQ ID NO: 3 - DNA, 497 bp, Artificial Sequence
  synthetic nucleotide sequence - Guayule Rep c34262
tagcaaactaccgaagacgatcgtttttcttcaacttcttcatcatccttgtctcaaaccgatactaccgaagtgcgcatatcgtcgtacgacgaaaaattcgaaaagaaagactgggaggaagaagtttaaagaaacaagacaccccgtgtatcggggtgtcaggttaagaaacgggtccaaatgggtaagcgaagtgcgtgagccgagtacaaaacatagaatatggttgggaacgtttcccactccggaaatggcggctagggcttatgatgcagcaactctagctttacgtggcgatgactcaccacttaactttagtgactcagcccgttcgattaaacgggctgtttcgaattcggatagtgatattaaacaagctgctttagaggcagmyctkcgcrkttgggccaagatgtcatgacattgagtcatcttctttggtagggtgttgttcggaggaaaaaggtggcggaaacatggcggtacctgcgcgg

SEQ ID NO: 4 - protein, 131 aa, Artificial Sequence
  synthetic peptide sequence - Guayule Rep c34262
SKLPKTIVFLQLLHHPCLKPILPKCAYRRTTKNSKRKTGRKKFKETRHPVYRGVRLRNGSKWVSEVREPSTKHRIWLGTFPTPEMAARAYDAATLALRGDDSPLNFSDSARSIKRAVSNSDSDIKQAALEA

SEQ ID NO: 5 - DNA, 627 bp, Artificial Sequence
  synthetic nucleotide sequence - Guayule Contig1228
  misc_feature (609)..(609): n is A, C, G or T
  misc_feature (613)..(614): n is A, C, G or T
  misc_feature (617)..(617): n is A, C, G or T
tagcaaactaccgaagacgatcgttttcttcaacttcttcatcatccttgtctcaaaccgatactaccgaagtgcgcatatcgtcgtcgacgaaaaattacgaaaagaaagactgggaggaagaagtttaaagaaacaagacaccccgtgtatcggggtgtcaggttaagaaacgggtccaaatgggtaagcgaagtgcgtgagccgagtacaaaacatagaatatggttgggaacgtttcccactccggaaatggcggctagggcttatgatgcagcaactctagctttacgtggcgatgactcaccacttaactttagtgactcagcccgttcgattaaacgggctgtttcgaattcggatagtgatattaaacaagctgctttagaggcagctcttgcatttgggccaagatgtcatgacattgagtcatcttctttggtagggtgttgttcggaggaaaaaggtggcggaaacatggcggtggaggtggcggatacgagttttgtggatgaagaggcgttgtttaatatgcctggtttttataatagcatggcggagcggtttggtgattacaccaccagggatgaaaaagggttttgactgganctgnncgnacctgcgcgg

SEQ ID NO: 6 - protein, 199 aa, Artificial Sequence
  synthetic peptide sequence - Guayule Contig1228
QTTEDDRFLQLLHHPCLKPILPKCAYRRRRKITKRKTGRKKFKETRHPVYRGVRLRNGSKWVSEVREPSTKHRIWLGTFPTPEMAARAYDAATLALRGDDSPLNFSDSARSIKRAVSNSDSDIKQAALEAALAFGPRCHDIESSSLVGCCSEEKGGGNMAVEVADTSFVDEEALFNMPGFYNSMAERFGDYTTRDEKGF

SEQ ID NO: 7 - DNA, 649 bp, Artificial Sequence
  synthetic nucleotide sequence - Guayule Contig10686
ccgcgcaggtaacaagacacccccgtgtatcggggtgtcaggttaagaaacggggtccaaatgggtaagcgaagtgcgtgagccgagtacaaaacatagaatatggtgggaacgtttcccactccggaaatggcggctagggcttatgatgcagcaactctagctttacgtggcgatgactcaccacttaactttagtgactcagcccgttcgattaaacgggctgtttcgaattcggatagtgatattaaacaagctgctttagaggcagctcttgcatttgggccaagatgtcatgacattgagtcatcttctttggtagggtgttgttcggaggaaaaaggtggcggaaacatggcggtggaggtggcggatacgagttttgtggatgaagaggcgttgtttaatatgcctggtttttataatagcatggcggagggtttggtgattacaccaccagggatgaaaaagggttttgactggagtgatgacattgattcggatatcgatttaactctctggaggtattagacataaacagtgacggaaccagaatattgtaatagaatggttacactataattttttgcatttgattagccagagggtcaagtgaccatttggacccactttggttccacctgcgcgg

SEQ ID NO: 8 - protein, 142 aa, Artificial Sequence
  synthetic peptide sequence - Guayule Contig10686
MVGTFPTPEMAARAYDAATLALRGDDSPLNFSDSARSIKRAVSNSDSDIKQAALEAALAFGPRCHDIESSSLVGCCSEEKGGGNMAVEVADTSFVDEEALFNMPGFYNSMAEGLVITPPGMKKGFDWSDDIDSDIDLTLWRY

SEQ ID NO: 9 - DNA, 699 bp, Artificial Sequence
  synthetic nucleotide sequence - Cold-induced transcript PBC003G09
caacaatcattactcataattaatggctacctttatccacttcaactctccgtacacttcactatcagttgtaaacactccaactgaatattcttgttcaacttccgactgctacagcaccggagcaagcttctccgacgaggaggtcgtcatgctggcgtcacaaaacccgaagaagcgggccgggaggaagaagtttagggagacacgacacccggtgtaccggggtgtccggcggagggattccggcaagtgggtgtgtgaggtaagagagcccaataagaaaacaagggtgtggttggggacgtacccaactgctgacatggcggcacgggctcatgacgtggcggctttggctatgcgggggcgatccgcgtgtttgaattttgctgactcgttgtggcggttgccgcttccggagtcgagtaatgtgaaggatattcaaaaggcggcggtgaaggcggctgaggcttttaggccggtggaatctgatgtgacgctgacggaggaaaggaaagagttgagtgaaaatgtgttttatgcggatgatgagagtgattttgggatgcaagggattcttgctgacatggccgaaggattgatggtaccgccacctcggacttttgggtttgaggatgacgtggatatttgtgttgacgcgccattgtggagtttttgaatttaggctttgactacatt

SEQ ID NO: 10 - protein, 218 aa, Artificial Sequence
  synthetic peptide sequence - Cold-induced transcript PBC003G09
MATFIHFNSPYTSLSVVNTPTEYSCSTSDCYSTGASFSDEEVVMLASQNPKKRAGRKKFRETRHPVYRGVRRRDSGKWVCEVREPNKKTRVWLGTYPTADMAARAHDVAALAMRGRSACLNFADSLWRLPLPESSNVKDIQKAAVKAAEAFRPVESDVTLTEERKELSENVFYADDESDFGMQGILADMAEGLMVPPPRTFGFEDDVDICVDAPLWSF

SEQ ID NO: 11 - DNA, 721 bp, Artificial Sequence
  synthetic nucleotide sequence - Cold-induced transcript PBC086F05
cacacaacaatcattactcataactaatggctacctttatccaattcaactctccgtacacttcactgtcagctgaaaatactccaactgaatattcttgctcaatttccgactgctacagcaccggagcaagcttttccgacgaggaggtcgtcatgctggcgtcacaaaacccgaagaagcgagccgggaggaagaagttcagggagacacgtcacccggtgtaccggggtgtccggcggagggattccggcaagtgggtgtgtgaggtaagagagcccaataagaaaacaagggtgtggttggggacgtacccaactgctgacatggcggcacgtgctcatgacgtggcggctttggcgatgcggggccggtccgcgtgtttgaattttgctgactcgttgtggcggttaccgcttccggagtcgagtaatgtgaaggatattcagaaggcggcggagaaggcggctgaggcttttaggccggtggagtctgatgtgacgttgatgaaggaaaggaaagagttaaatgaaaatgtgttttatgatgtggatgatgaaagtgttttggggatgcaagggattcttgctgacatggcagaaggattgatggtaccgccacctcggactgtcgggtttgaggatgacttggatttttgtgttgacgcgtcattgtggagttttttgaataaaggctttgactacattagagtgaggttgat

SEQ ID NO: 12 - protein, 231 aa, Artificial Sequence
  synthetic peptide sequence - Cold-induced transcript PBC086F05
MATFIQFNSPYTSLSAENTPTEYSCSISDCYSTGASFSDEEVVMLASQNPKKRAGRKKFRETRHPVYRGVRRRDSGKWVCEVREPNKKTRVWLGTYPTADMAARAHDVAALAMRGRSACLNFADSLWRLPLPESSNVKDIQKAAEKAAEAFRPVESDVTLMKERKELNENVFYDVDDESVLGMQGILADMAEGLMVPPPRTVGFEDDLDFCVDASLWSFLNKGFDYIRVRL

SEQ ID NO: 13 - DNA, 873 bp, Artificial Sequence
  synthetic TR46994 consensus nucleotide sequence
ccagtcgacagcatgtcccattaacagccgccaacgcacacccctcattcatcacccccctccttaaatacccactcatcatccactccacaaattcaattcctccaccaccactattattattattattatgggtgcacaacatcaccacaacactgattgttcatccagccgaaaaaaccaaattcataacaacaaaccataccgaggaatccgtatgcgcaagtgggggaagtgggttgccgagattcgtgagccgaataagcgctcacgaatctggctcggctcctactcttctcccgtagccgcggcccgcgcatacgacacggccgtgttttatctgcgcgggccgtcggctcggcttaattttcctgacttggttttggatgaccgcgccgacctttgtgacctttctgcggcttacatccggaaaaaggccacggaggtcggcgcgaaggtggacgcgctccagaaccagaccggaggcacgcgccaccactcgcccgaaagctattcgggtcgggtttgtttgaacacggatttgaatgagtatccgattccggaatcttctgatgggaattaacggtgagatgacgggaaggctatttgtttatatgttggttgacggcgttaatggacggagagttggataggctcttgttttttattttcttttttctttttaaatattaattggcggtcgggtcgggtcaagttgggttaggaatatatgtttcattgtatgaaaacaaaagaggaatggaaaattctgctacttttgttcttttgttgtttgtgtttgaattataagcaacatacaattgatatcagtatgaatacacgtacatacgtgcgtatgtatatgttttaacc

SEQ ID NO: 14 - protein, 150 aa, Artificial Sequence
  synthetic TR46994 polypeptide sequence
MGAQHHHNTDCSSSRKNQIHNNKPYRGIRMRKWGKWVAEIREPNKRSRIWLGSYSSPVAAARAYDTAVFYLRGPSARLNFPDLVLDDRADLCDLSAAYIRKKATEVGAKVDALQNQTGGTRHHSPESYSGRVCLNTDLNEYPIPESSDGN

SEQ ID NO: 15 - DNA, 916 bp, Artificial Sequence
  synthetic TR78450 consensus nucleotide sequence
  misc_feature (539)..(545): n is A, C, T or G
tgtcctcacacgcagacaaaaaactatatatagttcatcctttagatattattcacttaaaacaaaaccaaaacacttcactcacaacttgcaaataataaactataacaattcactgaacaaactacaaccgaaaacaaattatgctacttatggacaactctttagcttctgattgcagcaccgggaaactaatgctcgcttcgcaaaacccgaagaagcgagcggggagaacgaagttcaaagagactcgacacccggtttaccggggagtgaggataagaaactccggaaaatgggtgtgtgaggtgagagaaccaaataagaaatcgagagtgtggctggggacatacccgactgccgaaatggcggcccgagcacatgatgtggcggttttggcaatgaggggacggtcggcttgtttaaattttgctgactcggtttggcggctgccagtacccgagtccagcaatgtgaaagatatacaaaaggcggctgcggaggcggccgaggcgttccgagagacggaggatattgcnnnngnngtggaggtggtggtggtggaagcgaatgaggtgccggaagtcgtggtttatgcagatgaggaggagatgtttggaatgccgggatttattgccagcatggcggaaggactcatggtgtcgccacctcagatggtggggtatgctaactttgtgaataatgtggatttttgggaagacttgtctttatggagtttttagggtgttatattgttttcttattttagagtaagatacattttgtttgtttaggtaatggcttaaacctttttgtacacaagccaatattgaagtacatatatatcttcgaatagagatcaatcttttatttaacatagtagttaataatagttaagaatgatatgtgattgctt

SEQ ID NO: 16 - protein, 199 aa, Artificial Sequence
  synthetic R78450 polypeptide sequence
  MOD_RES (133)..(134): Xaa is any amino acid
MLLMDNSLASDCSTGKLMLASQNPKKRAGRTKFKETRHPVYRGVRIRNSGKWVCEVREPNKKSRVWLGTYPTAEMAARAHDVAVLAMRGRSACLNFADSVWRLPVPESSNVKDIQKAAAEAAEAFRETEDIAXXVEVVVVEANEVPEVVVYADEEEMFGMPGFIASMAEGLMVSPPQMVGYANFVNNVDFWEDLSLWSF

(19) United States
(12) Patent Application Publication
(10) Pub. No.: US 2018/0127767 A1
(43) Pub. Date: May 10, 2018

(54) INDUCTION OF LATEX ACCUMULATION IN RUBBER-PRODUCING SHRUBS

(71) Applicant: THE REGENTS OF THE UNIVERSITY OF CALIFORNIA, Oakland, CA (US)

(72) Inventors: Henrik Vibe SCHELLER, Millbrae, CA (US); Solomon STONEBLOOM, Alameda, CA (US)

(21) Appl. No.: 15/564,191

(22) PCT Filed: Apr. 1, 2016
(86) PCT No.: PCT/US2016/025695

§ 371 Date: Oct. 3, 2017

Related U.S. Application Data
(60) Provisional application No. 62/142,838, filed on Apr. 3, 2015.

(51) Int. Cl. C12N 15/82; C07K 14/415; C12N 5/04; C08L 7/02; A01H 5/00

(52) U.S. Cl. CPC C12N 15/8261; C07K 14/415; A01H 5/00; C08L 7/02; C12N 5/04

(57) ABSTRACT

The invention provides methods of engineering guayule plants to increase latex production. Specifically, the invention provides a method of engineering guayule plants with gene constructs that allow expression of master regulators of stress responses under non-stress conditions, the method comprising: introducing an expression cassette into the guayule plant, wherein the expression cassette comprises a polynucleotide encoding a dehydration response element binding protein/C-repeat binding factors (DREB/CBF) or DEAR [DREB and C-terminal EAR (ethylene response factor associated amphiphilic repression) motif] transcription factor operably linked to a promoter, such as a stem-specific promoter or an alcohol-inducible promoter. The invention additionally provides plants engineered in accordance with the invention and methods of using the plants to produce latex.

### CROSS-REFERENCES TO RELATED APPLICATIONS

This application claims priority to U.S. Provisional Application Nos. 62/142,838, filed Apr. 3, 2015, the entire content of which is incorporated in its entirety herein for all purposes.

### STATEMENT AS TO RIGHTS TO INVENTIONS MADE UNDER FEDERALLY SPONSORED RESEARCH AND DEVELOPMENT

This invention was made with government support under Contract No. DE-AC02-05CH11231 awarded by the U.S. Department of Energy. The government has certain rights in this invention.

### BACKGROUND OF THE INVENTION

Natural rubber is made from the latex exudate of the Para rubber tree (Hevea brasiliensis) and is used extensively in many industrial and commercial applications. While synthetic rubber (polyisoprene) is capable of substituting for natural rubber in some applications, the strength and thermal stability of natural, enzymatically polymerized rubber are critical in applications such as aviation and trucking tires. Currently, rubber is produced nearly exclusively in Southeast Asia. Economically viable rubber production from Hevea is dependent on inexpensive labor in developing countries and is threatened by a fungal rust that has prevented all efforts at large-scale production of rubber in the Americas. New sources of natural rubber would be advantageous for improving price and supply stability of this important industrial material. The North American desert shrub Guayule (Parthenium argentatum) was used to produce rubber by the Aztecs in northern Mexico and was cultivated widley during WWII when the supply of rubber from Malaysia was cut-off by the Japanese Pacific Blockade (Hammond & Polhamus, Research on guayule (Parthenium argentatum), 1942-1959, 1965). However, rubber production in Guayule remains uncompetitive with production in Hevea.

Yields of latex from guayule are relatively poor, as breeding of commercial varieties for increased production has been complicated by an apomyctic mode of reproduction and difficulties in evaluating latex yields in individual plants. Latex accumulation in guayule is largely dependent upon environmental factors, resulting in highly variable yields and restricting the areas where guayule can be cultivated for latex production. Previous studies of latex production show that latex production is promoted by cold and water restriction in guyaule.

A well characterized family of transcription factors regulates acclimation to cold temperatures and drought in plants, the CBFs. Overexpression of CBF transcription factors has been shown to functionally induce cold and drought tolerance when constituitively expressed in a number of plant species. Many of the metabolic changes observed following cold acclimation are observed in CBF-overexpressing Arabidopsis plants (Gilmour et al., Plant Physiol. 124:1854-1865, 2000).

Prior attempts to increase latex production in guayule focused on the overexpression of isoprenoid biosynthetic enzymes thought to be rate limiting. This strategy has not been successful to date: overexpression of these enzymes increased metabolic flux into the mevalonate pathway, however latex production was not significantly increased (see, Dong et al., Industrial Crops & Products 46:15-24, 2013.

As noted above, latex production in guayule is induced under cold and/or drought stress. Thus, reasonable yields cannot be achieved unless such stress occurs. On the other hand too much stress is obviously also disadvantageous. This invention provides for improved latex production in guayule without subjecting the plants to stress conditions.

### BRIEF SUMMARY OF THE INVENTION

This invention is based, in part, on the discovery of a strategy for increasing latex production in guayule without exposure to cold temperatures or water restriction by overexpression of CBF transcription factors in guayule; which produces metabolic changes typically accompanied by cold or drought.

In one aspect, the invention provides a method of engineering guayle plants with gene constructs that allows expression of master regulators of stress responses under non-stress conditions. In one embodiment, a nucleic acid encoding a transcription factor (TF) such as a CBF TF, e.g., CBF4, (either a heterologous gene or a guayule ortholog) is placed under the control of a constitutive promer, e.g., a 35S promoter and used to genetically modify guayule plants. This results in the constitutive expression of the TF. In some embdoiments, the nucleic acid encoding the TF is operably linked to a tissue-specific promoter, such as a stem cell promoter or a leaft promoter, e.g., RbcS or Lhc2 promoter. In some embodiments, the nucleic acid encoding the TF is operably linked to an inducible promoter so that latex production is induced by the application of the inducing agent, e.g., ethanol using an alcohol-inducible promoter.

Guayule plants modified in accordance with the invention have increased latex production relative to wildtype guayule plants that are not modified to overexpress a CBF transcription factor. Such plants can be used for the extraction of latex.

### BRIEF DESCRIPTION OF THE DRAWINGS

FIG. 1: The phylogeny of previously sequenced Guayule orthologs of CBF/DREB transcription factors as well as TR78450 and TR46994. TR78450 is similar to the Arabidopsis CBF transcription factors, while TR46994 is similar to the DEAR transcription factors. The predicted amino acid sequences were aligned using CLUSTALw. The phylogenetic tree was constructed using the neighbor joining algorithm. Numbers indicate bootstrap values for each node.

FIG. 2: Relative expression of guayule transcription factors TR78450 and TR46994 in tissue samples from rubber-producing, cold-treated and control non-rubber producing plants. CL: cold-treated leaves, CS: cold-treated stems, UTL: untreated leaves, UTS: untreated stems. Brackets indicate one standard deviation, n=3 for each data point.

### DETAILED DESCRIPTION OF THE INVENTION

### I. Definitions

As used herein, the term “dehydration response element binding protein/C-repeat binding factors” or “DREB/CBF” transcription factor is used interchangeably with “CBF transcription factor” and refers to a member of a family of transcription factors that target genes containing cold- and drought-responsive DNA regulatory sequences designated as C-repeat (CRT)/dehydration-responsive elements (DREs), which have a conserved core sequence (Baker et al., 1994; Yamaguchi-Shinozaki and Shinozaki, 1994). The transcription factors are AP2/EREBP domain-containing transcription factors in clade 3 of the AP2/ERF family. Arabidopsis encodes a family of cold- and drought-responsive transcriptional factors known as CBF1, CBF2, CBF3, and CBF4 (also called DREB1B, DREB1C, DREB1A, and DREB1D, respectively). These transcription factors are part of the CBF/DREB cold-response pathway (Jaglo-Ottosen et al., 1998; Zhang et al., 2004) and regulate a common set of genes involved in responses to abiotic stresses (see, e.g., Park et al. Regulation of the Arabidopsis CBF regulon by a complex low-temperature regulatory network. The Plant Journal (2015)). CBF1, CBF2 and CBF3 are cold responsive transcription factors while CBF4 is largely responsive to cold-stress; however, all of the CBF/DREB transcription factors have been shown to regulate the expression of genes involved in responses to drought and cold stresses. Homologs of CBF/DREB transcription factors from plants other than Arabidopsis may not be identifyable as homologs of a specific Arabidopsis CBF1, CBF2, CBF3, or CBF4 CBF/DREB gene as the Arabidopsis CBF1, CBF2 and CBF3 genes appear to be recent duplications of a single ancestral gene. As used herin, the “CBF/DREB” family of transcription factors refers to transcription factors binding a C-repeat (CRT)/Dehydration-responsive Element (DRE). This CRT/DRE core sequence was found to be present in the promoter regions of many cold-inducible genes, such as rd29A and cor15a (Maruyama et al., 2004). The CBF/DREB transcription factors are members of DREB subfamily A-1 of ERF/AP2 domain-containing proteins (Nakano, Genome-Wide Analysis of the ERF Gene Family in Arabidopsis and Rice. Plant Physiol (2006) vol. 140 (2) pp. 411-432). ERF/AP2 domain characteristics are described, e.g., in Nakano, supra. In the present invention, CBF/DREB genes in plant species other than Arabidopsis are considered to be genes encoding protein that have a CRT/DRE binding domain and have at least 60%, 65%, 70%, 75%, 80%, 85%, 90%, or 95% identity, or greater, with to an Arabidposis CBF1, CBF2, CBF3, or CBF4 protein sequence. In some embodiments, a CCBF/DREB transcription factor may have at least 60%, 65%, 70%, 75%, 80%, 85%, 90%, or 95% identity, or greater, to a reference CBF transcription factor sequence from a plant species other than Arabidopsis. An illustrative guayule CBF transcription factor polypeptide sequence is provided in SEQ ID NO:16.

As used herein, the “DEAR” family of transcription factors refers to transcription factors that contain an N-terminal DREB1/CBF (dehydration-responsive element binding protein 1/C-repeat binding factor) domain and a C-terminal EAR (ethylene response factor-associated amphiphilic repression) motif. The DEAR transcription factors are members of DREB subfamily A-5 of ERF/AP2 domain-containing proteins (Nakano, 2006, supra). The DEAR TFs appear to mediate cross-talk between drought, cold and pathogen responses (Tsutsui et al. DEAR1, a transcriptional repressor of DREB protein that mediates plant defense and freezing stress responses in Arabidopsis. J Plant Res (2009) vol. 122 (6) pp. 633-643.). As used herein, the DEAR family of transcription factors in plant species other than Arabidopsis refers to ERF/AP2 domain-containing transcription factors possessing at least 50%, or at least 60%, 65%, 70%, 75%, 80%, 85%, 90%, or 95%, identity to the Arabidopsis DEAR transcription factors. In some embodiments, a DEAR transcription factor may have at least 50%, or at least 60%, 65%, 70%, 75%, 80%, 85%, 90%, or 95% identity, or greater, to a reference DEAR transcription factor sequence from a plant species other than Arabidopsis. An illustrative guayule DEAR transcription factor polypeptide sequence is provided in SEQ ID NO:14.

As used herein, the term “transcription factor that regulates the production of components of a biosynthetic pathway” or “master transcription factor” refers to a transcription factor that regulates expression of one or of multiple genes in a biosynthetic pathway.

The term “downstream target,” when used in the context of a downstream target of a transcription factor that regulates a component of a latex production pathway refers to a gene or protein whose expression is directly or indirectly regulated by the transcription factor.

The terms “increased latex production” refers to an increase in the amount of latex produced by a guayule plant genetically modified in accordance with the invention in comparison to a wildtype guayule plant or a guayule plant that has not been recombinantly modified to express a DREB/CBF or DEAR transcription factor (e.g., a CBF4 transcription factor or DEAR1 transcription factor). A guayule plant with increased latex production typically produces at last least 10%, 20%, 30%, 40%, 50%, 60%, 70%, 80%, or 90% or greater compared to a wildtype plant grown under non-stress conditions.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer. These cis-acting sequences typically interact with proteins or other biomolecules to carry out (turn on/off, regulate, modulate, etc.) gene transcription. Promoters are located 5′ to the transcribed gene, and as used herein, typically include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is referred to by the name of the gene for which it naturally regulates expression. Reference to a promoter by name includes a wildtype, native promoter as well as variants of the promoter that retain the ability to induce expression. Reference to a promoter by name is not restricted to a particular plants species, but also encompasses a promoter from a corresponding gene in other plant species.

A “constitutive promoter” in the context of this invention refers to a promoter that is capable of initiating transcription in nearly all cell types, whereas a “cell type-specific promoter” or “tissue-specific promoter” initiates transcription only in one or a few particular cell types or groups of cells forming a tissue. In some embodiments, a promoter is tissue-specific if the transcription levels initiated by the promoter in a tissue are at least 2-fold, 3-fold, 4-fold, 5-fold, or 10-fold, or higher as compared to the transcription levels initiated by the promoter in an unrelated tissue. In some embodiments, the promoter is a “strong” tissue-specific promoter that initiates transcription levels that result in at least 5-fold or at least 10-fold increased expression of a transcript compared to another tissue.

A polynucleotide is “heterologous” to an organism or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

As used herein, “recombinant” used in reference to a cell or vector, refers to a cell or vector that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. Thus, “recombinant” or “engineered” or “non-naturally occurring” when used with reference to a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. The term encompasses progeny of cells that have been manipulated using recombinant techniques.

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “expression cassette” or “DNA construct” or “expression construct” refers to a nucleic acid construct that, when introduced into a host cell, results in transcription and/or translation of an RNA or polypeptide, respectively. Antisense or sense constructs that are not or cannot be translated are expressly included by this definition. Expression constructs can include multiple elements, e.g., a promoter, an enhancer, a transcription terminator, an origin of replication, a chromosomal integration sequence, 5′ and 3′ untranslated regions, or an intronic sequence, which are involved in transcriptional regulation, and the like. In the case of both expression of transgenes and suppression of endogenous genes one of skill will recognize that the inserted polynucleotide sequence need not be identical, but may be only substantially identical to a sequence of the gene from which it was derived. One example of an expression cassette is a polynucleotide construct that comprises a polynucleotide sequence encoding a DREB/CBF protein operably linked to a heterologous promoter. In some embodiments, an expression cassette comprises a polynucleotide sequence encoding a DREB/CBF protein that is targeted to a position in a plant genome such that expression of the polynucleotide sequence is driven by a promoter that is present in the plant

The term “plant” as used herein can refer to a whole plant or part of a plant, e.g., seeds, and includes plants of a variety of ploidy levels, including aneuploid, polyploid, diploid and haploid. The term “plant part,” as used herein, refers to shoot vegetative organs and/or structures (e.g., leaves, stems and tubers), branches, roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, and plant tissue (e.g., vascular tissue, ground tissue, and the like), as well as individual plant cells, groups of plant cells (e.g., cultured plant cells), protoplasts, plant extracts, and seeds. In the present invention guayule plants are genetically modified.

### DREB/CBF and DEAR Nucleic Acid Sequences

The invention employs various routine recombinant nucleic acid techniques.

Generally, the nomenclature and the laboratory procedures in recombinant DNA technology described below are those well known and commonly employed in the art. Many manuals that provide direction for performing recombinant DNA manipulations are available, e.g., Sambrook & Russell, Molecular Cloning, A Laboratory Manual (3rd Ed, 2001); and Current Protocols in Molecular Biology (Ausubel, et al., John Wiley and Sons, New York, 2009, supplements through 2014).

In the present invention, a guayule plant is genetically modified to constiutively express a DREB/CBF or DEAR transcription factor, e.g., CBF4 or DEAR1, or to express the DREB/CBF or DEAR transcription factor, e.g., CBF4 or DEAR1, under the control of a tissue-specific promoter, e.g., a leaf promoter, or an inducible promoter, such as an ethanol-inducible promoter, to increase latex production.

DREB/CBF transcription factors are well known in the art and have been extensively studied in plants. CBF genes (for C-repeat/DRE binding factor) encode proteins that interact with a specific cis-acting element of certain plant promoters. (U.S. Pat. Nos. 5,296,462 and 5,356,816; Yamaguchi-Shinozaki, et al., (1994) The Plant Cell 6:251-264; Baker, et al., (1994) Plant Mol. Biol. 24:701-713; Jiang, et al., (1996) Plant Mol. Biol. 30:679-684). CBF proteins comprise a CBF-specific domain and an AP2 domain and have been identified in various species, including Arabidopsis (Stockinger, et al., (1997) Proc. Natl. Acad. Sci. 94:1035-1040; Liu, et al., (1998) Plant Cell 10:1391-1406); Brassica napus, Lycopersicon esculentum, Secale cereale, and Triticum aestivum (Jaglo, et al., (2001) Plant Phys. 127:910-917) and Brassica juncea, Brassica oleracea, Brassica rapa, Raphanus sativus, Glycine max, and Zea mays (U.S. Pat. Nos. 6,417,428; 7,253,000 and 7,317,141, incorporated by reference). The AP2 domain is highly conserved among CBF genes, and some species share an additional conserved region bracketing the AP2 domains. (Jaglo, et al., (2001) Plant Phys. 127:910-917). As noted above, DREB/CBF polypeptide and nucleic acid sequences are known in many plants. Additional illustrative DREB/CBF polypeptide and nucleic acid sequences are provided in U.S. Patent Application Publication No. 20050076412, which is incorporated by reference.

DRE/CRT (Dehydration Response Element/C-Repeat) cis elements function in response to stress, e.g., cold and drought, and have been identified in numerous plant species, including Arabidopsis, barley, Brassica, citrus, cotton, eucalyptus, grape, maize, melon, pepper, rice, soy, tobacco, tomato and wheat. The DRE/CRT elements comprise a core binding site, A/GCCGAC, recognized by the trans-activating factors known as DREB1 (DRE-Binding) and CBF (C-Repeat Binding Factor). Secondary structure in proximity to the cis element, and/or multiple cis factors appear to be additional components necessary for stress-inducible expression. (For reviews, see, Agarwal, et al., (2006) Plant Cell Rep 25:1263-1274; Yamaguchi-Shinozaki and Shinozaki, (2005) Trends in Plant Science 10(2):88-94).

Overexpression of CBF in plants has been shown to improve tolerance to drought, cold, and/or salt stress (Jaglo-Ottosen, et al., (1998) Science 280:104-106; Kasuga, et al., (1999) Nature Biotechnology 17:287-291; Hsieh, et al., (2002) Plant Phys. 129:1086-1094; Hsieh, et al., (2002) Plant Phys. 130:618-626; Dubouzet, et al., (2003) Plant J. 33:751-763; Thomashow, U.S. Pat. No. 5,929,305).

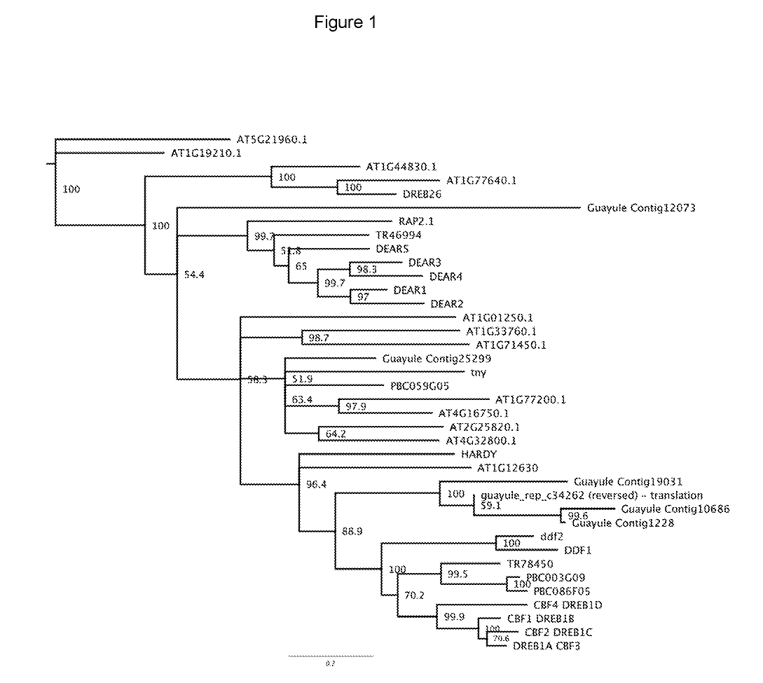

In some embodiments, a nucleic acid encoding a DREB/CBF transcription factor is an ortholog of a DREB/CBF transcription factor noted above that is obtained from guayule or a closely related plant, such as sunflower. Orthologs of DREB/CBF transcription factors involved in cold acclimation from guayule or the closely related sunflower genome can be identified by BLAST searches of publicly available EST and RNAseq databases (such as those available at http://compgenomics.ucdavis.edu/). These CBF transcription factor-like genes can be identified in transcript assemblies available from the Compositae Genomics Project (http://compgenomics.ucdavis.edu/cgp_wd_assemblies.php#35935) as well as in supplemental data from Ponciano et al., Phytochemistry 79:57-66, 2012. With respect to CBF/DREB genes in guayule, a small clade of genes can be defined in the available guayule transcriptome data with greater than 45% total pairwise amino acid identity with the Arabidopsis CBF/DREB and DDF transcription factors (Kang et al. Plant Science 180:634-641, 2011). These guayule CBF orthologs form a clade with the Arabidopsis CBF/DREB and DDF, as shown in FIG. 1. DREB/CBF TFs involved in responses to cold stress in guayule can also be identified by transcriptome analysis of mRNAs from cold-treated and control guayule tissues, such as the guayule CBF/DREB TF TR78450. Accordingly, the CBF orthologs indicated in the CBF/DREB clade in FIG. 1 can be used in the modification of latex production in guayule in accordance with the present invention. Additional AP2 family transcription factors such as guayule contigs 12073, TR46994, and PBC059G05 in FIG. 1 are less similar to the CBF/DREB transcription factors (21% pairwise identity).

In other embodiments a nucleic acid encoding an AP2/ERF domain containing DEAR family protein is an ortholog obtained from guayule or a close relative such as sunflower. Orthologs of DEAR transcription factors can be identified by BLAST searches of publicly available EST and RNAseq databases, or found in de novo transcript assemblies of RNAseq data from cold-treated guayule plants such as TR46994 in FIG. 1. A small clade of DEAR transcription factors can be identified with a pairwise predicted amino acid identity of greater than 50%. The DEAR family TF ortholog TR46994 indicated in FIG. 1 can be used in the modification of latex production in guayule in accordance with the present invention.

In some embodiments, a guayule plant is genetically modified to express a CBF/DREB polypeptide that comprises a sequence of any one of SEQ ID NOS:2, 4, 6, 8, 10, or 12, or a variant that comprises at least 70% identity, typically at least 75% identity, at least 80% identity, at least 85% identity, at least 90% identity, at least 95% identity, or at least 96%, at least 97%, at least 98%, or at least 99% to any one of SEQ ID NOS:2, 4, 6, 8, 10, or 12. In some embodiments, a guayule plant is genetically modified to express a CBF/DREB polypeptide that comprises a sequence of SEQ ID NO:16, or a variant that comprises at least 70% identity, typically at least 75% identity, at least 80% identity, at least 85% identity, at least 90% identity, at least 95% identity, or at least 96%, at least 97%, at least 98%, or at least 99% to SEQ ID NO:16.

In some embodiments, a guayule plant is genetically modified to express a DEAR polypeptide that comprises a sequence of SEQ ID NO:14, or a variant that comprises at least 70% identity, typically at least 75% identity, at least 80% identity, at least 85% identity, at least 90% identity, at least 95% identity, or at least 96%, at least 97%, at least 98%, or at least 99% to SEQ ID NO:14. Methods and computer programs for the alignment are well known in the art. The term “identity” or “homology” as used here refers to the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software. Examples include BLAST or BLAST 2.0 with default parameters.

Isolation or generation of DREB/CBF or DEAR polynucleotides can be accomplished by a number of well-known techniques. In some embodiments, oligonucleotide probes based on the sequences disclosed here can be used to identify the desired polynucleotide in a cDNA or genomic DNA library from a desired plant species. Probes may be used to hybridize with genomic DNA or cDNA sequences to isolate homologous genes in the same or different plant species.

Alternatively, the nucleic acids of interest can be amplified from nucleic acid samples using routine amplification techniques. For instance, PCR may be used to amplify the sequences of the genes directly from mRNA, from cDNA, from genomic libraries or cDNA libraries. PCR and other in vitro amplification methods may also be useful for other purposes, e.g., nucleic acid sequencing, to obtain a desired fragment of a polynucleotide of interest.

### Preparation of Recombinant Vectors

To use isolated sequences in the above techniques, recombinant DNA constructs suitable for transformation of guayule cells are prepared. Techniques for preparing such constructs are well known and described in the technical and scientific literature. For example, a DNA sequence encoding a DREB/CBF polypepitde can be combined with transcriptional and other regulatory sequences which will direct the transcription of the sequence from the gene in the intended cells, e.g., guayule stem cells. In some embodiments, an expression vector that comprises an expression cassette that comprises the DREB/CBF gene further comprises a promoter operably linked to the DREB/CBF gene. In other embodiments, a promoter and/or other regulatory elements that direct transcription of the DREB/CBF gene are endogenous to the plant and an expression cassette comprising the DREB/CBF ene is introduced, e.g., by homologous recombination, such that the heterologous DREB/CBF gene is operably linked to an endogenous promoter and is expression driven by the endogenous promoter. In some embodiments, a promoter that drives expression of the DREB/CBF gene may be a promoter of a gene involved in latex production in guayule. Any number of promoters may be used to drive expression of the DREB/CBF gene, including either constitutive or inducible, or tissue-specific promoters.

### Tissue-Specific Promoters

In some embodiments, a plant promoter to direct expression of a DREB/CBF gene in a specific tissue is employed (tissue-specific promoters). Tissue specific promoters are transcriptional control elements that are only active in particular cells or tissues at specific times during plant development.

Tissue-specific promoters include promoters that initiate transcription only (or primarily only) in certain tissues, such as vegetative tissues, cell walls, roots or leaves. A variety of promoters specifically active in vegetative tissues, such as leaves, stems, and roots are known. For example, promoters controlling patatin, the major storage protein of the potato tuber, can be used (see, e.g., Kim, Plant Mol. Biol. 26:603-615, 1994; Martin, Plant J. 11:53-62, 1997). The ORF13 promoter from Agrobacterium rhizogenes that exhibits high activity in roots can also be used (Hansen, Mol. Gen. Genet. 254:337-343, 1997). Other useful vegetative tissue-specific promoters include: the tarin promoter of the gene encoding a globulin from a major taro (Colocasia esculenta L. Schott) corm protein family, tarin (Bezerra, Plant Mol. Biol. 28:137-144, 1995); the curculin promoter active during taro corm development (de Castro, Plant Cell 4:1549-1559, 1992) and the promoter for the tobacco root-specific gene TobRB7, whose expression is localized to root meristem and immature central cylinder regions (Yamamoto, Plant Cell 3:371-382, 1991).

In some embodiments, leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier, FEBS Lett. 415:91-95, 1997). A ribulose bisphosphate carboxylase promoters expressed almost exclusively in mesophyll cells in leaf blades and leaf sheaths at high levels (e.g., Matsuoka, Plant J. 6:311-319, 1994), can be used. Another leaf-specific promoter is the light harvesting chlorophyll a/b binding protein gene promoter (see, e.g., Shiina, Plant Physiol. 115:477-483, 1997; Casal, Plant Physiol. 116:1533-1538, 1998). The Arabidopsis thaliana myb-related gene promoter (Atmyb5) (Li, et al., FEBS Lett. 379:117-121 1996), is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize (e.g., Busk et al., Plant J. 11:1285-1295, 1997) can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, (e.g., Di Laurenzio, et al., Cell 86:423-433, 1996; and, Long, et al., Nature 379:66-69, 1996); can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto, Plant Cell. 7:517-527, 1995). Also useful are knl-related genes from maize and other species which show meristem-specific expression, (see, e.g., Granger, Plant Mol. Biol. 31:373-378, 1996; Kerstetter, Plant Cell 6:1877-1887, 1994; Hake, Philos. Trans. R. Soc. Lond. B. Biol. Sci. 350:45-51, 1995). For example, the Arabidopsis thaliana KNAT1 promoter (see, e.g., Lincoln, Plant Cell 6:1859-1876, 1994) can be used.

Other examples of promoters are secondary cell wall promoters such as IRX1, IRX3, IRX5, IRX8, IRX9, IRX14, IRX7, IRX10, GAUT13, or GAUT14 promoters.

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

### Constitutive Promoters

A promoter, or an active fragment thereof, can be employed which will direct expression of a nucleic acid encoding a fusion protein of the invention, in all or most transformed cells or tissues, e.g. as those of a regenerated plant. Such promoters are referred to herein as “constitutive” promoters and are active under most environmental conditions and states of development or cell differentiation. Examples of constitutive promoters include those from viruses which infect plants, such as the cauliflower mosaic virus (CaMV) 35S transcription initiation region (see, e.g., Dagless, Arch. Virol. 142:183-191, 1997); the 1′- or 2′-promoter derived from T-DNA of Agrobacterium tumefaciens (see, e.g., Mengiste supra (1997); O'Grady, Plant Mol. Biol. 29:99-108, 1995); the promoter of the tobacco mosaic virus; the promoter of Figwort mosaic virus (see, e.g., Maiti, Transgenic Res. 6:143-156, 1997); ubiquitin promoers, actin promoters, such as the Arabidopsis actin gene promoter (see, e.g., Huang, Plant Mol. Biol. 33:125-139, 1997); alcohol dehydrogenase (Adh) gene promoters (see, e.g., Millar, Plant Mol. Biol. 31:897-904, 1996); ACT11 from Arabidopsis (Huang et al., Plant Mol. Biol. 33:125-139, 1996), Cat3 from Arabidopsis (GenBank No. U43147, Zhong et al., Mol. Gen. Genet. 251:196-203, 1996), the gene encoding stearoyl-acyl carrier protein desaturase from Brassica napus (Genbank No. X74782, Solocombe et al., Plant Physiol. 104:1167-1176, 1994), GPc1 from maize (GenBank No. X15596, Martinez et al., J. Mol. Biol. 208:551-565, 1989), Gpc2 from maize (GenBank No. U45855, Manjunath et al., Plant Mol. Biol. 33:97-112, 1997), other transcription initiation regions from various plant genes known to those of skill. See also Holtorf, “Comparison of different constitutive and inducible promoters for the overexpression of transgenes in Arabidopsis thaliana,” Plant Mol. Biol. 29:637-646, 1995).

### Inducible Promoters

In some embodiments, an inducible promoter is used. Examples of inducible promoters include plant promoters that are inducible upon exposure to plant hormones, such as auxins. For example, the invention can use the auxin-response elements E1 promoter fragment (AuxREs) in the soybean (Glycine max L.) (Liu, Plant Physiol. 115:397-407, 1997); the auxin-responsive Arabidopsis GST6 promoter (also responsive to salicylic acid and hydrogen peroxide) (Chen, Plant J. 10: 955-966, 1996); the auxin-inducible parC promoter from tobacco (Sakai, 37:906-913, 1996); or a plant biotin response element (Streit, Mol. Plant Microbe Interact. 10:933-937, 1997). Other examples of useful promoters include alcohol-inducible promoters, e.g., an ethanol inducible promoter.

Plant promoters inducible upon exposure to chemicals reagents that may be applied to the plant, such as herbicides or antibiotics, are also useful for expressing a DRB/CBF gene in accordance with the invention. For example, the maize In2-2 promoter, activated by benzenesulfonamide herbicide safeners, can be used (De Veylder, Plant Cell Physiol. 38:568-577, 19997); application of different herbicide safeners induces distinct gene expression patterns, including expression in the root, hydathodes, and the shoot apical meristem. A DREB/CBF coding sequence can also be under the control of, e.g., a tetracycline-inducible promoter, such as described with transgenic tobacco plants containing the Avena sativa L. (oat) arginine decarboxylase gene (Masgrau, Plant J. 11:465-473, 1997); or, a salicylic acid-responsive element (Stange, Plant J. 11:1315-1324, 1997; Uknes et al., Plant Cell 5:159-169, 1993); Bi et al., Plant J. 8:235-245, 1995).

Further examples of inducible regulatory elements include copper-inducible regulatory elements (Mett et al., Proc. Natl. Acad. Sci. USA 90:4567-4571, 1993); Furst et al., Cell 55:705-717, 1988); tetracycline and chlor-tetracycline-inducible regulatory elements (Gatz et al., Plant 2:397-404, 1992); Roder et al., Mol. Gen. Genet. 243:32-38, 1994); Gatz, Meth. Cell Biol. 50:411-424, 1995); ecdysone inducible regulatory elements (Christopherson et al., Proc. Natl. Acad. Sci. USA 89:6314-6318, 1992; Kreutzweiser et al., Ecotoxicol. Environ. Safety 28:14-24, 1994); heat shock inducible regulatory elements (Takahashi et al., Plant Physiol. 99:383-390, 1992; Yabe et al., Plant Cell Physiol. 35:1207-1219, 1994; Ueda et al., Mol. Gen. Genet. 250:533-539, 1996); and lac operon elements, which are used in combination with a constitutively expressed lac repressor to confer, for example, IPTG-inducible expression (Wilde et al., EMBO J. 11:1251-1259, 1992). An inducible regulatory element useful in the transgenic plants of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

### Additional Promoters

Further, endogenous promoters from genes in the latex biosynthesis pathway can be used to drive expression of transcription factors activating the biosynthesis of rubber, producing an artificial positive feedback loop to drive latex production strongly once it has been naturally induced. Promoters of known rubber biosynthesis genes previously identified in guayule and known to be highly expressed in latex producing tissue, such as SRPP, farnesyl-phosphate synthase and allene oxide synthase can be used for this purpose (see, e.g., Ponciano et al., Phytochemistry 79:57-66, 2012, which is incorporated by reference). Additional promoters from Rubber Elongation Factor, hydroxymethylglutaryl CoA synthase, Cis-prenyltransferase and allene oxide synthase can also be employed.

### Additional Embodiments for Expressing CREB/CBF

In another embodiment, the CREB/CBF polynucleotide is expressed through a transposable element. This allows for constitutive, yet periodic and infrequent expression of the constitutively active polypeptide. The invention also provides for use of tissue-specific promoters derived from viruses including, e.g., the tobamovirus subgenomic promoter (Kumagai, Proc. Natl. Acad. Sci. USA 92:1679-1683, 1995); the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer, Plant Mol. Biol. 31:1129-1139, 1996).

A vector comprising CERB/CBF nucleic acid sequences will typically comprise a marker gene that confers a selectable phenotype on the cell to which it is introduced. Such markers are known. For example, the marker may encode antibiotic resistance, such as resistance to kanamycin, G418, bleomycin, hygromycin, and the like.

CREB/CBF nucleic acid sequences of the invention are expressed recombinantly in plant cells as described. As appreciated by one of skill in the art, expression constructs can be designed taking into account codon usage frequencies. Codon usage frequencies can be tabulated using known methods (see, e.g., Nakamura et al. Nucl. Acids Res. 28:292, 2000). Codon usage frequency tables are also available in the art (e.g., from the Codon Usage Database at the internet site www.kazusa.or.jp/codon/.)

Additional sequence modifications may be made that are also known to enhance gene expression in a plant. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence may also be modified to avoid predicted hairpin secondary mRNA structures.

As an example illustrating generation of a construct encoding a DREB/CBF for expression in guayule, primers are designed to amplify a polynucleotide encoding a latex production-regulating transcription factor, e.g., a DREB/CBF transcription factor comprising an amino acid sequence of SEQ ID NO:2, 4, 6, 8, 10, or 12 for cloning into an Agrobacterium binary vector for plant transformation and protein expression, such as pCAMBIA2300. Vectors are constructed to drive expression of the transcription factors in guayule, e.g., using a constitutive promoter such as the CaMV 35s or a ubiquitin or rubisco small subunit promoter.

### Production of Transgenic Plants

As detailed herein, the present invention provides for transgenic plants comprising recombinant expression cassettes for expressing a DREB/CBF transcription factor. It should be recognized that the term “transgenic plants” as used here encompasses the plant or plant cell in which the expression cassette is introduced as well as progeny of such plants or plant cells that contain the expression cassette, including the progeny that have the expression cassette stably integrated in a chromosome.

Once an expression cassette comprising a polynucleotide encoding a a DREB/CBF transcription factor has been constructed, standard techniques may be used to introduce the polynucleotide into a plant in order to modify gene expression. See, e.g., protocols described in Ammirato et al. (1984) Handbook of Plant Cell Culture--Crop Species. Macmillan Publ. Co. Shimamoto et al. (1989) Nature 338:274-276; Fromm et al. (1990) Bio/Technology 8:833-839; and Vasil et al. (1990) Bio/Technology 8:429-434.

Transformation and regeneration of plants is known in the art, and the selection of the most appropriate transformation technique will be determined by the practitioner. Suitable methods may include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens mediated transformation. Transformation means introducing a nucleotide sequence in a plant in a manner to cause stable or transient expression of the sequence. Examples of these methods in various plants include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

In one embodiment, a CBF/DREB construct in accordance with the invention can be transformed into Guayule using an Agrobacterium co-cultivation technique (see, e.g., Dong et al., Industrial Crops & Products 46:15-24, 2013 and Dong et al., Plant Cell Rep 25:26-34, 2006). In brief, portions of Guayule leaf are co-cultivated with Agrobacterium tumefactiens strains carrying the construct of interest on a binary vector for plant transformation. After co-cultivation with the agrobacterium, undifferentiated callus tissue is cultivated from the leaves and transgenic calli are selected. Transgenic plants are then regenerated from the callus tissue. For example, binary vectors carrying Kanamycin resistance or phosphomannose-isomerase genes for selection of transgenic plants can be used for plant transformation with latex production regulating constructs. For phosphomannose isomerase selection, transgenic calli can be selected by providing 20 g/l mannose as the only carbon source, e.g., as described by Wang et al., Plant Cell Rep 19:654-660, 2000. Transgenic calli can be transferred to regeneration medium for rooting then transferred to grow on soil. Transgenic plants can then be propagated by cuttings prior to testing for latex accumulation with and without an inducer, if an inducible promoter is used, such as an ethanol-inducible promoter, or cold induction of latex production by previously described methods such as accelerated solvent extraction.

Transformed plant cells derived by any of the above transformation techniques can be cultured to regenerate a whole plant that possesses the transformed genotype and thus the desired phenotype. Such regeneration techniques may involve manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally, e.g., in Klee et al. Ann. Rev. of Plant Phys. 38:467-486, 1987.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

A guayule plant that is genetically modified to express a DREB/CBF polypeptide can be identified using any known assay, including analysis of RNA, protein, or latex content compared to a wildtype guayule plant. With respect to this aspect of the invention, the plants have enhanced latex levels.

A guayule plant genetically modified in accordance with the invention can be used to obtain latex. Methods of extracting latex from guayule are known in the art. For example, rubber is found primarily in the bark and is released during processing. Plant material comprising bark, e.g., the whole plant or branches, are homogenized in an aqueous extraction medium. The rubber particles obtained from the parenchyma cells of the guayule plant are thereby released into the solution to create an aqueous suspension comprising the particles. The rubber particles, which have a specific gravity of slightly less than 1, can then be purified from the homogenate using a series of centrifugation steps and/or flotation with creaming agents. This process results in natural rubber latex with very little remaining cytoplasmic or soluble protein components. Examples of methods of extracting latex are provided in e.g., WO2007081376; U.S. Patent Application Publication Nos. 20110021743, 20070265408, and 20060106183; U.S. Pat. Nos. 5,717,050, 5,580,942, and 6,054,525, each of which are incorporated by referenced. Purification of latex is also described in Cornish K. and J. L. Brichta. 2002. Purification of hypoallergenic latex from guayule. p. 226-233, 2002, In: J. Janick and A. Whipkey (eds.), Trends in new crops and new uses. ASHS Press, Alexandria, Va., also incorporated by reference, and references cited therein.

### Examples

To better characterize the expression and sequence of Guayule CBF/DREB genes, the transcriptomes of rubber producing and non-rubber producing plants were sequenced. First, rubber production was induced in Guayule AZ2 cultivar plants by placing plants in a growth chamber simulating winter conditions for 4 weeks. Conditions were 13 h 5° C. night and 11 h 25° day. Messenger RNA was then extracted from stem and leaf tissues of rubber producing, cold-induced plants as well as stem and leaf tissues of control, un-induced plants. The transcriptomes were sequenced on Illumina Miseq and Hiseq2500 sequencing platforms following directional RNA sequencing library preparation. Sequence data were assembled using the Trinity algorithm (Haas et al., Nat Protoc 8: 1494-1512, 2013) and analyzed for differential expression using edgeR (Robinson et al., Bioinformatics 26, 139-140, 2010). tBLASTn searches against these transcriptome assemblies identified two DREB/CBF transcription factor genes that were significantly upregulated following the induction of rubber biosynthesis: TR46994 and TR78450. The consensus nucleotide and predicted polypeptide sequences are provided in SEQ ID NOs:13-16.

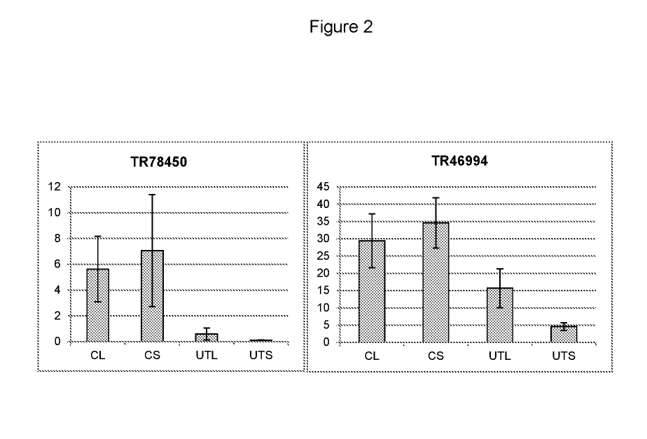

TR78450 is most similar to CBF4 (FIG. 1) and is upregulated 7.5 fold in rubber producing stems over non-rubber producing stems (FIG. 2). TR46994 is most similar to the DEAR family of stress and freezing tolerance regulating transcription factors, which were been previously shown to have a role in modulating responses to biotic and abiotic stress (FIG. 1) (Tsutsui et al., J Plant Res 122, 633-643, 2009). TR46994 is upregulated 58.8 fold in cold treated stems compared with cold-treated leaves.

To demonstrate that these genes are useful for increasing rubber production in Guayule, the genes are overexpressed in Guayule plants either constitutively, with an inducible promoter, or using a promoter to create a positive feedback loop once rubber biosynthesis has initiated naturally.

To further verify the role of the genes in rubber production, the promoter region of genes involved in the production of rubber in Guayule and also up-regulated in rubber producing tissues are cloned into binary vectors for agrobacterium transformation, driving the expression of reporter genes such as GFP, YFP, RFP or GUS(beta-glucuronidase). The promoters of genes such as the Rubber Elongation Factor, Hydroxymethylglutaryl CoA synthase, Cis-prenyltransferase and Allene Oxide Synthase aree used. Similar promoter-reporter experiments have been previously described (Hellens et al., Plant Methods 1:13, 2005). Rubber promoter constructs will be transiently introduced into Nicotiana benthamiana or lettuce leaves, with constructs overexpressing TR46994 and TR76450. Increased activity of rubber-biosynthetic gene promoters when TR46994 and TR76450 are expressed.

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference in their entirety for all purposes.

Illustrative Guayule CBF/DREB sequences of the CBF/DREB clade of FIG. 1:

Guayule Contig 19031 (SEQ ID NO: 1):
GAGTTAACAAAATGGAAACTATTGAATACGATGGTTCTTCTTCGATCTCT
TCATCATCATCGTCTCAAACCGAGACTACTGAACGACTACCCACACCATC
GTCAACAAACCACTCGAAAAGAAAATCCGGAAGGAAGAAGTTTAAAGAAA
CGAGACACCCGGTCTATAGAGGGGTCCGGTTGAGAAACGGGTCTAAATGG
NNNTGCGNNGTTCGTGAGCCTAGTAAGAAATCAAGAATTTGGTTAGGAAC
ATTTCCAACTCCGGAAATGGCAGCTAGGGCTTATGATGCAGCAACCCTAG
CCTTGCATGGTGATGACTCACCACTTAACTTTGCTGACTCGGCCCGTCTA
ATTAGACGGGCCAAGTCAACTTCTGCTTCTGATGTTCAGGAGGCAGCCCT
TGAGGCGGCTCTTGCATTTGGGTCAAATCATCATGTTGACCCTAAGCCGT
CTTCTTCATCTTCAGCGTGTTTTCAGATGGAGAAAGGTGAGGAAAACTTG
ACAGTTGATGTGGCGCATACATCATTTGTGGACGAAGAGTTGTTGTTTAA
TACGCCGAGTTTTTATAACAGTATGGCGGAGGGATTGGTGATTACACCAC
CGGGTATGAAAAAAGGGTTTGATTGGAGTGATGATTGTGCCTCCAATATG
GATTTGACTCTATACCTGCGCGG
Guayule Contig 19031 protein sequence (SEQ ID
NO: 2):
METIEYDGSSSISSSSSSQTETTERLPTPSSTNHSKRKSGRKKFKETRHP
VYRGVRLRNGSKW?C?VREPSKKSRIWLGTFPTPEMAARAYDAATLALHG
DDSPLNFADSARLIRRAKSTSASDVQEAALEAALAFGSNHHVDPKPSSSS
SACFQMEKGEENLTVDVAHTSFVDEELLFNTPSFYNSMAEGLVITPPGMK
KGFDWSDDCASNMDLTLYLR
Guayule Rep c34262 (SEQ ID NO: 3):
TAGCAAACTACCGAAGACGATCGTTTTTCTTCAACTTCTTCATCATCCTT
GTCTCAAACCGATACTACCGAAGTGCGCATATCGTCGTACGACGAAAAAT
TCGAAAAGAAAGACTGGGAGGAAGAAGTTTAAAGAAACAAGACACCCCGT
GTATCGGGGTGTCAGGTTAAGAAACGGGTCCAAATGGGTAAGCGAAGTGC
GTGAGCCGAGTACAAAACATAGAATATGGTTGGGAACGTTTCCCACTCCG
GAAATGGCGGCTAGGGCTTATGATGCAGCAACTCTAGCTTTACGTGGCGA
TGACTCACCACTTAACTTTAGTGACTCAGCCCGTTCGATTAAACGGGCTG
TTTCGAATTCGGATAGTGATATTAAACAAGCTGCTTTAGAGGCAGMYCTK
CGCRKTTGGGCCAAGATGTCATGACATTGAGTCATCTTCTTTGGTAGGGT
GTTGTTCGGAGGAAAAAGGTGGCGGAAACATGGCGGTACCTGCGCGG
Guayule Rep c34262 predicted amino acid sequence
(SEQ ID NO: 4):
SKLPKTIVFLQLLHHPCLKPILPKCAYRRTTKNSKRKTGRKKFKETRHPV
YRGVRLRNGSKWVSEVREPSTKHRIWLGTFPTPEMAARAYDAATLALRGD
DSPLNFSDSARSIKRAVSNSDSDIKQAALEA
Guayule Contig1228 (SEQ ID NO: 5):
TAGCAAACTACCGAAGACGATCGTTTTCTTCAACTTCTTCATCATCCTTG
TCTCAAACCGATACTACCGAAGTGCGCATATCGTCGTCGACGAAAAATTA
CGAAAAGAAAGACTGGGAGGAAGAAGTTTAAAGAAACAAGACACCCCGTG
TATCGGGGTGTCAGGTTAAGAAACGGGTCCAAATGGGTAAGCGAAGTGCG
TGAGCCGAGTACAAAACATAGAATATGGTTGGGAACGTTTCCCACTCCGG
AAATGGCGGCTAGGGCTTATGATGCAGCAACTCTAGCTTTACGTGGCGAT
GACTCACCACTTAACTTTAGTGACTCAGCCCGTTCGATTAAACGGGCTGT
TTCGAATTCGGATAGTGATATTAAACAAGCTGCTTTAGAGGCAGCTCTTG
CATTTGGGCCAAGATGTCATGACATTGAGTCATCTTCTTTGGTAGGGTGT
TGTTCGGAGGAAAAAGGTGGCGGAAACATGGCGGTGGAGGTGGCGGATAC
GAGTTTTGTGGATGAAGAGGCGTTGTTTAATATGCCTGGTTTTTATAATA
GCATGGCGGAGCGGTTTGGTGATTACACCACCAGGGATGAAAAAGGGTTT
TGACTGGANCTGNNCGNACCTGCGCGG
Guayule Contig1228 amino acid sequence (SEQ ID
NO: 6):
QTTEDDRFLQLLHHPCLKPILPKCAYRRRRKITKRKTGRKKFKETRHPVY
RGVRLRNGSKWVSEVREPSTKHRIWLGTFPTPEMAARAYDAATLALRGDD
SPLNFSDSARSIKRAVSNSDSDIKQAALEAALAFGPRCHDIESSSLVGCC
SEEKGGGNMAVEVADTSFVDEEALFNMPGFYNSMAERFGDYTTRDEKGF*
Guayule Contig10686: (SEQ ID NO: 7)
CCGCGCAGGTAACAAGACACCCCCGTGTATCGGGGTGTCAGGTTAAGAAA
CGGGGTCCAAATGGGTAAGCGAAGTGCGTGAGCCGAGTACAAAACATAGA
ATATGGTGGGAACGTTTCCCACTCCGGAAATGGCGGCTAGGGCTTATGAT
GCAGCAACTCTAGCTTTACGTGGCGATGACTCACCACTTAACTTTAGTGA
CTCAGCCCGTTCGATTAAACGGGCTGTTTCGAATTCGGATAGTGATATTA
AACAAGCTGCTTTAGAGGCAGCTCTTGCATTTGGGCCAAGATGTCATGAC
ATTGAGTCATCTTCTTTGGTAGGGTGTTGTTCGGAGGAAAAAGGTGGCGG
AAACATGGCGGTGGAGGTGGCGGATACGAGTTTTGTGGATGAAGAGGCGT
TGTTTAATATGCCTGGTTTTTATAATAGCATGGCGGAGGGTTTGGTGATT
ACACCACCAGGGATGAAAAAGGGTTTTGACTGGAGTGATGACATTGATTC
GGATATCGATTTAACTCTCTGGAGGTATTAGACATAAACAGTGACGGAAC
CAGAATATTGTAATAGAATGGTTACACTATAATTTTTTGCATTTGATTAG
CCAGAGGGTCAAGTGACCATTTGGACCCACTTTGGTTCCACCTGCGCGG
Guayule Contig10686 amino acid sequence (SEQ ID
NO: 8):
MVGTFPTPEMAARAYDAATLALRGDDSPLNFSDSARSIKRAVSNSDSDIK
QAALEAALAFGPRCHDIESSSLVGCCSEEKGGGNMAVEVADTSFVDEEAL
FNMPGFYNSMAEGLVITPPGMKKGFDWSDDIDSDIDLTLWRY*
Cold-induced transcript PBC003G09 (SEQ ID NO: 9):
CAACAATCATTACTCATAATTAATGGCTACCTTTATCCACTTCAACTCTC
CGTACACTTCACTATCAGTTGTAAACACTCCAACTGAATATTCTTGTTCA
ACTTCCGACTGCTACAGCACCGGAGCAAGCTTCTCCGACGAGGAGGTCGT
CATGCTGGCGTCACAAAACCCGAAGAAGCGGGCCGGGAGGAAGAAGTTTA
GGGAGACACGACACCCGGTGTACCGGGGTGTCCGGCGGAGGGATTCCGGC
AAGTGGGTGTGTGAGGTAAGAGAGCCCAATAAGAAAACAAGGGTGTGGTT
GGGGACGTACCCAACTGCTGACATGGCGGCACGGGCTCATGACGTGGCGG
CTTTGGCTATGCGGGGGCGATCCGCGTGTTTGAATTTTGCTGACTCGTTG
TGGCGGTTGCCGCTTCCGGAGTCGAGTAATGTGAAGGATATTCAAAAGGC
GGCGGTGAAGGCGGCTGAGGCTTTTAGGCCGGTGGAATCTGATGTGACGC
TGACGGAGGAAAGGAAAGAGTTGAGTGAAAATGTGTTTTATGCGGATGAT
GAGAGTGATTTTGGGATGCAAGGGATTCTTGCTGACATGGCCGAAGGATT
GATGGTACCGCCACCTCGGACTTTTGGGTTTGAGGATGACGTGGATATTT
GTGTTGACGCGCCATTGTGGAGTTTTTGAATTTAGGCTTTGACTACATT
Cold-induced transcript PBC003G09 amino acid
sequence (SEQ ID NO: 10):
MATFIHFNSPYTSLSVVNTPTEYSCSTSDCYSTGASFSDEEVVMLASQNP
KKRAGRKKFRETRHPVYRGVRRRDSGKWVCEVREPNKKTRVWLGTYPTAD
MAARAHDVAALAMRGRSACLNFADSLWRLPLPESSNVKDIQKAAVKAAEA
FRPVESDVTLTEERKELSENVFYADDESDFGMQGILADMAEGLMVPPPRT
FGFEDDVDICVDAPLWSF*
Cold-induced transcript PBC086F05 (SEQ ID NO: 11):
CACACAACAATCATTACTCATAACTAATGGCTACCTTTATCCAATTCAAC
TCTCCGTACACTTCACTGTCAGCTGAAAATACTCCAACTGAATATTCTTG
CTCAATTTCCGACTGCTACAGCACCGGAGCAAGCTTTTCCGACGAGGAGG
TCGTCATGCTGGCGTCACAAAACCCGAAGAAGCGAGCCGGGAGGAAGAAG
TTCAGGGAGACACGTCACCCGGTGTACCGGGGTGTCCGGCGGAGGGATTC
CGGCAAGTGGGTGTGTGAGGTAAGAGAGCCCAATAAGAAAACAAGGGTGT
GGTTGGGGACGTACCCAACTGCTGACATGGCGGCACGTGCTCATGACGTG
GCGGCTTTGGCGATGCGGGGCCGGTCCGCGTGTTTGAATTTTGCTGACTC
GTTGTGGCGGTTACCGCTTCCGGAGTCGAGTAATGTGAAGGATATTCAGA
AGGCGGCGGAGAAGGCGGCTGAGGCTTTTAGGCCGGTGGAGTCTGATGTG
ACGTTGATGAAGGAAAGGAAAGAGTTAAATGAAAATGTGTTTTATGATGT
GGATGATGAAAGTGTTTTGGGGATGCAAGGGATTCTTGCTGACATGGCAG
AAGGATTGATGGTACCGCCACCTCGGACTGTCGGGTTTGAGGATGACTTG
GATTTTTGTGTTGACGCGTCATTGTGGAGTTTTTTGAATAAAGGCTTTGA
CTACATTAGAGTGAGGTTGAT
Cold-induced transcript PBC086F05 amino acid
sequence (SEQ ID NO: 12):
MATFIQFNSPYTSLSAENTPTEYSCSISDCYSTGASFSDEEVVMLASQNP
KKRAGRKKFRETRHPVYRGVRRRDSGKWVCEVREPNKKTRVWLGTYPTAD
MAARAHDVAALAMRGRSACLNFADSLWRLPLPESSNVKDIQKAAEKAAEA
FRPVESDVTLMKERKELNENVFYDVDDESVLGMQGILADMAEGLMVPPPR
TVGFEDDLDFCVDASLWSFLNKGFDYIRVRL
TR46994 consensus nucleotide sequence (SEQ ID
NO: 13)
CCAGTCGACAGCATGTCCCATTAACAGCCGCCAACGCACACCCCTCATTC
ATCACCCCCCTCCTTAAATACCCACTCATCATCCACTCCACAAATTCAAT
TCCTCCACCACCACTATTATTATTATTATTATGGGTGCACAACATCACCA
CAACACTGATTGTTCATCCAGCCGAAAAAACCAAATTCATAACAACAAAC
CATACCGAGGAATCCGTATGCGCAAGTGGGGGAAGTGGGTTGCCGAGATT
CGTGAGCCGAATAAGCGCTCACGAATCTGGCTCGGCTCCTACTCTTCTCC
CGTAGCCGCGGCCCGCGCATACGACACGGCCGTGTTTTATCTGCGCGGGC
CGTCGGCTCGGCTTAATTTTCCTGACTTGGTTTTGGATGACCGCGCCGAC
CTTTGTGACCTTTCTGCGGCTTACATCCGGAAAAAGGCCACGGAGGTCGG
CGCGAAGGTGGACGCGCTCCAGAACCAGACCGGAGGCACGCGCCACCACT
CGCCCGAAAGCTATTCGGGTCGGGTTTGTTTGAACACGGATTTGAATGAG
TATCCGATTCCGGAATCTTCTGATGGGAATTAACGGTGAGATGACGGGAA
GGCTATTTGTTTATATGTTGGTTGACGGCGTTAATGGACGGAGAGTTGGA
TAGGCTCTTGTTTTTTATTTTCTTTTTTCTTTTTAAATATTAATTGGCGG
TCGGGTCGGGTCAAGTTGGGTTAGGAATATATGTTTCATTGTATGAAAAC
AAAAGAGGAATGGAAAATTCTGCTACTTTTGTTCTTTTGTTGTTTGTGTT
TGAATTATAAGCAACATACAATTGATATCAGTATGAATACACGTACATAC
GTGCGTATGTATATGTTTTAACC
TR46994 polypeptide sequence (SEQ ID NO: 14)
MGAQHHHNTDCSSSRKNQIHNNKPYRGIRMRKWGKWVAEIREPNKRSRIW
LGSYSSPVAAARAYDTAVFYLRGPSARLNFPDLVLDDRADLCDLSAAYIR
KKATEVGAKVDALQNQTGGTRHHSPESYSGRVCLNTDLNEYPIPESSDG
N*
TR78450 consensus nucleotide sequence (SEQ ID
NO: 15)
TGTCCTCACACGCAGACAAAAAACTATATATAGTTCATCCTTTAGATATT
ATTCACTTAAAACAAAACCAAAACACTTCACTCACAACTTGCAAATAATA
AACTATAACAATTCACTGAACAAACTACAACCGAAAACAAATTATGCTAC
TTATGGACAACTCTTTAGCTTCTGATTGCAGCACCGGGAAACTAATGCTC
GCTTCGCAAAACCCGAAGAAGCGAGCGGGGAGAACGAAGTTCAAAGAGAC
TCGACACCCGGTTTACCGGGGAGTGAGGATAAGAAACTCCGGAAAATGGG
TGTGTGAGGTGAGAGAACCAAATAAGAAATCGAGAGTGTGGCTGGGGACA
TACCCGACTGCCGAAATGGCGGCCCGAGCACATGATGTGGCGGTTTTGGC
AATGAGGGGACGGTCGGCTTGTTTAAATTTTGCTGACTCGGTTTGGCGGC
TGCCAGTACCCGAGTCCAGCAATGTGAAAGATATACAAAAGGCGGCTGCG
GAGGCGGCCGAGGCGTTCCGAGAGACGGAGGATATTGCNNNNGNNGTGGA
GGTGGTGGTGGTGGAAGCGAATGAGGTGCCGGAAGTCGTGGTTTATGCAG
ATGAGGAGGAGATGTTTGGAATGCCGGGATTTATTGCCAGCATGGCGGAA
GGACTCATGGTGTCGCCACCTCAGATGGTGGGGTATGCTAACTTTGTGAA
TAATGTGGATTTTTGGGAAGACTTGTCTTTATGGAGTTTTTAGGGTGTTA
TATTGTTTTCTTATTTTAGAGTAAGATACATTTTGTTTGTTTAGGTAATG
GCTTAAACCTTTTTGTACACAAGCCAATATTGAAGTACATATATATCTTC
GAATAGAGATCAATCTTTTATTTAACATAGTAGTTAATAATAGTTAAGAA
TGATATGTGATTGCTT
TR78450 polypeptide sequence SEQ ID NO: 16
MLLMDNSLASDC STGKLMLASQNPKKRAGRTKFKETRHPVYRGVRIRNS
GKWVCEVREPNKKSRVWLGTYPTAEMAARAHDVAVLAMIRGRSACLNFAD
SVWRLPVPESSNVKDIQKAAAEAAEAFRETEDIA??VEVVVVEANEVPEV
VVYADEEEMFGMPGFIASMAEGLMVSPPQMVGYANFVNNVDFWEDLSLWS
F*

1. A method of engineering a guayule plant to increase latex production, the method comprising:
introducing an expression cassette into the guayule plant, wherein the expression cassette comprises a polynucleotide encoding a DREB/CBF or DEAR transcription factor operably linked to a promoter and
culturing the plant under conditions in which the transcription factor is expressed.

2. The method of claim 1, wherein the transcription factor is a CBF4 transcription factor.

3. The method of claim 1, wherein the transcription factor is shown in the CBF/DREB Clade in FIG. 1.

4. The method of claim 1, wherein the transcription factor is a DEAR1 transcription factor.

5. The method of claim 1, wherein the transcription factor is shown in the DEAR Clade in FIG. 1.

6. The method of claim 1, wherein the promoter is a stem-specific promoter.

7. The method of claim 1, wherein the promoter is an inducible promoter.

8. The method of claim 7, wherein the inducible promoter is an alcohol-inducible promoter

9. A plant engineered by the method of claim 1, or a progeny of the plant.

10. A plant cell from the plant of claim 9.

11. Seed from the plant of claim 9.

12. A method of obtaining latex, the method comprising extracting latex from a plant engineered by the method of claim 1.
